(19) United States
(12) Patent Application Publication
(10) Pub. No.: US 2011/0092871 A1
(43) Pub. Date: Apr. 21, 2011

(54) WOUND DRESSING

(75) Inventors: Tomas Fabo, Molnlycke (SE); Ulf Johannison, Landvetter (SE); Dennis Hansson, Gunnilse (SE)

(73) Assignee: Molnlycke Health Care AB, Goteborg (SE)

(21) Appl. No.: 12/996,133

(22) PCT Filed: Jun. 16, 2009
(86) PCT No.: PCT/SE09/50746

§ 371 Date: Dec. 3, 2010

(30) Foreign Application Priority Data
Jun. 24, 2008 (SE) 08014904

(51) Int. Cl. A61L 15/62; A61L 15/22

(52) U.S. Cl. 602/46; 602/43

(57) ABSTRACT

The present invention relates to a wound dressing including an outer layer of air-impermeable material and means for attaching the dressing to the skin surrounding a wound, a pre-compressed body of resilient material, said body creating a sub-pressure after attaching the dressing to a wound. According to the invention the pre-compressed body is enclosed in a casing of air-tight material, whereby at least a portion of said casing is made of a soluble material, which dissolves after contact with liquid exudate.

### TECHNICAL FIELD

The present invention relates to wound dressing including an outer layer of air-impermeable material, means for attaching the dressing to the skin surrounding a wound, and a pre-compressed body of resilient material, said body creating a sub-pressure after attaching the dressing to a wound.

### BACKGROUND TO THE INVENTION

It has been shown to be beneficial for the healing of a wound if the wound bed is subjected to a sub-atmospheric pressure or sub-pressure. Wound dressings in which a sub-pressure is created in a space surrounding a wound bed are also known in the art, see for example U.S. Pat. No. 4,969,880, U.S. Pat. No. 5,645,081, U.S. Pat. No. 5,636,643, U.S. Pat. No. 6,855,135, W02006/025848, US2008/0082059 and US 2008/0004559. Such dressings, so called NPT-dressings (Negative Pressure Therapy), are usually connected to a pump-driven, often external source of sub-pressure by a tubing or the like. The external source of sub-pressure may be a stationary device or a device that the patient can bring with him. Depending on the tightness against leakage of air into the dressing, the external source may be driven continuously or intermittently. Sub-pressure also increases the rate of exudation and thereby improves cleaning of a wound. In addition, wound dressing systems with sub-pressure also effectively take care of the exudate and reduce the risk of leakage from the wound dressing to the wound environment. The wound exudate sucked up from the wound bed can be stored in an external container or a porous body included in the dressing. The application and handling of known sub-pressure dressings and relating devices are thus rather complicated and time-consuming and there is accordingly room for improvement. Furthermore, the tubing used in such dressings must be rigid enough to withstand the pressure difference between the sub-pressure and the atmospheric pressure essentially without change of sectional shape. Such tubing can therefore be irritating or damaging to the skin of a patient if it is pressed against the skin by external forces, such as affixing tape or tight clothing or if the patient is lying thereon. In addition to a complicated waste handling, there are also the above described disadvantages concerning patient comfort and skin damage when instead portable pumps (e.g. mechanical or electrical) are used by the patient. It would therefore be an advantage if such equipment could be avoided.

US 2008/0103462 A1 discloses a wound dressing composed of a self-expanding absorbent material encased in an outer barrier completely surrounding the absorbent material, the inner surface thereof containing openings and also a peripheral adhesive layer. In one embodiment said openings are open and in another embodiment said openings are closed by a dissolvable sealant. There is no indication in the document of how the dressing according to the second embodiment is manufactured, i.e. how the outer barrier is applied to the self-expanding material or how the sealant is applied to the openings in the inner surface thereof. A dressing according to the second embodiment would be complicated to manufacture.

The objective of the present invention to obtain a wound dressing including an outer layer of air-impermeable material, means for attaching the dressing to the skin surrounding a wound, and a pre-compressed body of resilient material, said body creating a sub-pressure after attaching the dressing to a wound which functions well and is easy to manufacture.

### SUMMARY OF THE INVENTION

This objective is accomplished by a wound dressing including an outer layer of air-impermeable material, means for attaching the dressing to the skin surrounding a wound and a pre-compressed body of resilient material, said body creating a sub-pressure after attaching the dressing to a wound, characterised in that the pre-compressed body is enclosed in a casing of air-tight material which is separate from the outer layer, whereby at least a portion of said casing is made of a soluble material, which dissolves after contact with liquid exudates. The pre-compressed body of such a wound dressing can be manufactured separately and then brought into a production line of usual type for manufacturing of wound dressings. The normal equipment used for such a production line need thus not be changed or supplemented with devices for compressing resilient bodies and the production rate in such a production line need not be slowed down by a compression step. Moreover, the enclosing of a compressed body in such a casing is easily accomplished. If the entire casing or at least the major part of the lower side thereof, i.e. the side intended to be turned against a wound bed during use of the dressing, is made of soluble material, a large area for exudate to enter the pre-compressed body exists which leads to a faster absorption of exudate than if exudate only can enter through openings in liquid impermeable layer and possibly also to a quicker start of the absorption process.

In a preferred embodiment, the pre-compressed body has the ability to directly or indirectly suck liquid from a wound bed. The pre-compressed body can then constitute the wound pad of the dressing or part of the wound pad, which can be constituted of several layers of which the pre-compressed body constitutes one of these layers.

The pre-compressed body is preferably made of resilient foam, for example polyurethane foam. Other foams can also be used, such as viscoelastic foam.

Preferably, the dressing is stored in a vacuum package.

Preferably, the casing is, at least on the side thereof, which is turned against a wound bed when the dressing is used, made of soluble material, which can consist of polyvinyl alcohol.

In a second embodiment, the pre-compressed body enclosed in its casing is disposed outside the outer layer of the dressing and the unit of pre-compressed body and casing is enclosed in a second layer of air-tight material, which is releasably attached to the outer layer in a region around an opening in the outer layer and the second layer.

The invention also relates to a combination of a first wound dressing including a wound pad and a cover layer of air-tight material and a second wound dressing of the type described above, which is attached on top of the first wound dressing in a region thereof surrounding a hole in the cover layer of the first wound dressing.

### BRIEF DESCRIPTION OF THE DRAWING

The invention will now be described with reference to the enclosed figures, of which;

FIG. 1 schematically discloses a cross-sectional view of a wound dressing according to a first preferred embodiment of the invention,

FIG. 2 schematically discloses a cross-sectional view of a wound dressing according to a second preferred embodiment of the invention, and

FIGS. 3 and 4 schematically illustrates the manufacture of a pre-compressed body.

### DESCRIPTION OF EMBODIMENTS

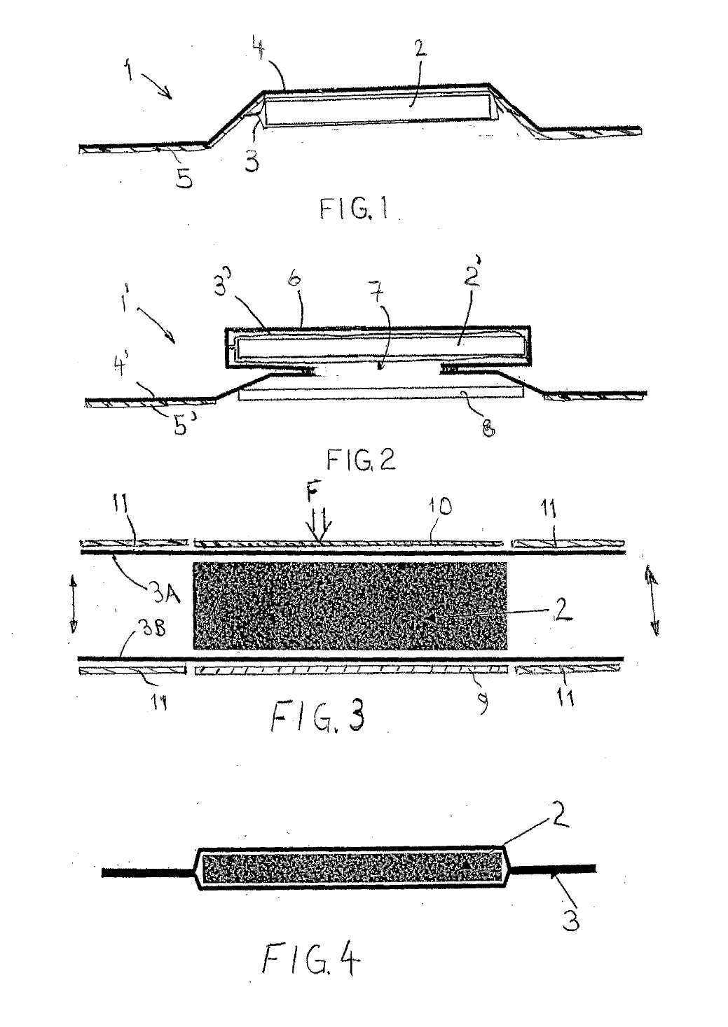

In FIG. 1 a cross-sectional view of a wound dressing 1 according to a first preferred embodiment is schematically disclosed. The dressing 1 includes a pre-compressed body 2 of compressible and resilient material, such as plastic foam. The body 2 is in a compressed state enclosed in an airtight casing 3 before it is disposed within the dressing 1, as is schematically shown in FIG. 1. Thereby, the pre-compression of body 2 is maintained during handling of body 2, storing of this body and dressings including such bodies and transport thereof. The air-tight casing 3 consists entirely or at least on a large part of the underside thereof, of a material which dissolves when in contact with liquid, for example a film of polyvinyl alcohol. The body 2 in its casing 3 is covered by an outer layer 4 of an airtight material, preferably a plastic film, which extends laterally beyond the circumference of body 2 in order to enable affixation of the dressing 1 to skin surrounding a wound bed. On the underside of film 4, i.e. the side turned against body 2, a coating of a skin-friendly adhesive 5 is applied at least in the portion of the film extending laterally outside of the circumference of body 2. The upper side of said casing 3 may preferably be attached to the underside of the outer layer 4 in one or more parts.

By “upper side” and “underside” relates to the position of the dressing shown in FIGS. 1 and 2, independent of where a dressing is attached to a patient. The “underside” is thus the side of the dressing turned against the skin of a patient when the dressing is used.

Before use of the dressing 1, the underside thereof is covered with a protection layer (not shown) for protecting the adhesive coating 5 before use, as is usual in the art. The protection layer is made of or coated with a substance that adheres poorly to the adhesive. Thus, the material used for the protection layer is depending on the adhesive used and all known material combinations of adhesive and protection layer can be used in the present invention. An example of such a combination is a silicone adhesive covered by a polyethylene layer or a paper layer having a polyethylene coating on the side thereof releasably attached to the silicone adhesive. The protection layer is removed from the dressing before the dressing is applied to a patient.

Moreover, if the covering plastic film 4 is very thin, e.g. thinner than 50 micrometer, a stiffening layer (not shown) in form of a frame around the edge regions of the portion of film 3 extending laterally outside of body 2 is removably attached to the top side of film 4. i.e. the side distal from the body 2 as also is usual in the art. The stiffening layer can be made of any material known in the art to be used for stiffening layers for film dressings or the like. Such stiffening layers are removed in connection with the application of a dressing to a wound.

When the wound dressing 1 has been attached to an exuding wound, the underside of or at least a major portion of the casing 4 will dissolve when the exudate comes into contact with the casing and the body 2 can start to expand due to its resiliency. The expanding body will create an expanding volume under the covering film and thereby create a sub-pressure in the fluidum between the wound surface and the covering film. When more exudate is entering into the wound dressing, the body will continue to expand. When the body has reached its fully expanded shape or is hindered to expand further by external mechanical forcers (such as e.g. from the plastic film 4 or the surrounding atmospheric pressure), no more sub-pressure will be generated by the body.

The adhesive coating 5 on the underside of film 4 should be such that the space delimited by a wound bed and the portion of film 4 covering the top and sides of body 2 is sealed against the environment so that air can not leak into this space. A coating of a silicone adhesive with a softness of at least 10 mm and applied in an amount of at least e.g. 50 g/m² is suitable for this purpose as is evident from WO 2006/075950, to which is referred to for further details.

In FIG. 2, a second preferred embodiment of a wound dressing 1′ according to the present invention is schematically shown. The wound dressing 1′ differs from the wound dressing 1 described with reference to FIG. 1 mainly in that the pre-compressed body 2′ is disposed outside the adhesively coated film 4′ instead of inside said film and that dressing 1′ also comprises a body 8 of absorbent material within the space delimited by the film 4′. Components in the wound dressing 1′ corresponding to similar components in the wound dressing 1 according to FIG. 1 are given the same reference numerals with the addition of a prime sign. In the second embodiment, the pre-compressed body 2′ is enclosed in an air- and liquid-tight envelope 6 which is connected to the interior of film 4′ by an opening or a channel 7. The envelope 6 can consist of a film of plastic material and has such a size that the pre-compressed body can expand therein to a desired degree without being obstructed by the envelope. Moreover, the envelope 6 and the plastic film 4′ are bonded to each other in a region around the channel 7 by any suitable means, such as glue bond or a weld bond. Also in this embodiment, the pre-compressed body 2′ is enclosed in a casing 3′ of which at least the underside or a major part thereof is constituted by a film of soluble material.

In a variant the envelope 6 can be sealed by a film of a soluble material covering the opening 7. In such a case, a separate casing 3′ for the pre-compressed body can be deleted.

When the absorbent body 8 is totally or locally saturated so that the underside of casing 3′ or piece of soluble material covering opening 7 comes into contact with absorbed exudate from a wound bed, the soluble material will dissolve and the body 2′ will start to expand thereby creating a sub-pressure.

In the wound dressing 1′, the body 8 can not be omitted since a body distancing the film 4′ from the wound bed when the dressing 1′ is subjected to the difference in pressure between atmospheric pressure and the sub-pressure created by the pre-compressed body 2′ is needed. In all other aspects, the components in the wound dressing 1′ can be the same as in the wound dressing 1 described with reference to FIG. 1 and the wound dressing 1′ will function in the same way.

In an alternative, the body 2′ and its envelope 6 is manufactured as a separate body which can be attached to a so called island dressing comprised of an adhesive coated thin film 3′ and a wound pad 8. In such a case the envelope 6 will on its underside, i.e. the side having an opening 7 covered by a piece of soluble material, have an adhesive region surrounding the opening 7 for attaching the envelope 6 to the film 4′. Furthermore, the film 4′ will have an opening corresponding to the opening 7 which preferably is covered by a removable piece of material. In such a case, the envelope 6 with its pre-compressed body 2′ needs not to be attached to film 4′ until the body 8 is saturated which means that a sub-pressure will be created immediately after attachment of the envelope 6 to the film 4′. An advantage of this alternative construction is that a used envelope 6 with a pre-compressed body 2′, that has expanded to a determined degree, can be substituted by a fresh envelope 6 having a fully pre-compressed body 2′ without having to change the under-lying body 8 and the film 4′.

It is of course possible to attach such a separate envelope 6 to a conventional island dressing without a pre-formed opening 7 by cutting an opening in such a conventional island dressing.

Due to the sub-pressure created when the wound dressing 1′ has been attached to skin surrounding a wound and when the liquid soluble material constituting the seal between body 2′ and the environment is broken in the attached wound dressing 1′, the bodies 2′,8 will be pressed tightly against each other and body 8 will be pressed tightly to the wound bed mainly by a pressure corresponding to the pressure difference between the atmospheric pressure and the sub-pressure created in the space above the wound bed. Exudate will then be drained from body 8 and sucked into body 2′ mainly due to the created sub-pressure but also by capillary forces. When, as preferred, the wound dressing 1′ is constructed so that body 2′ can drain body 8, the body 2′ is part of the wound pad of the wound dressing 1′.

The expansion of the body 2′ must develop unobstructed by the film 6 enclosing bodies 2′ when the wound dressing is attached to the skin surrounding a wound. The size of the film 6 is thus preferably such that it allows expansion of the body 2′ to a relaxed state. It is of course possible to choose a larger or intermediate size of the film 6 depending on desired properties of the wound dressing. A larger size can be chosen in order to ensure that the ability of the compressed resilient body 2′ to expand is used completely. An intermediate size can be used in order to indicate when the wound dressing 1 has to be changed in order to ensure that the wound is continuously subjected to a sub-pressure. The indication would then be that the wound dressing should be changed when folds or uneven parts no longer are present on the film 6.

The same size requirements are of course also applicable to the film 4 in the embodiment shown in FIG. 1. However, an intermediate size will not be chosen for envelope 4. In this respect, it is pointed out that although the underside of film 4 may have an adhesive coating on parts covering the body 2, the regions of the film which contains folds or the like due to the oversize of film 4 must of course be free of adhesive.

In the embodiment shown in FIG. 2, the body 8 is constituted by an absorbent material, such as a foam with open cells or a fibrous nonwoven or textile material made of absorbent fibres. However, it is not necessary that the body 8 is of absorbent material, it is enough that it is permeable for air and liquid. It can thus, for example, be constituted of a net of absorbent or non-absorbent material, a perforated plastic film or a nonwoven or textile material made of non-absorbent fibres.

An absorbent or non-absorbent body similar to body 8 in the second embodiment can of course also be present in the wound dressing 1 according to FIG. 1 placed between the pre-compressed body and the wound bed.

The materials used in a wound dressing according to the present invention are such that the dressing can be sterilized, for example by ethylene oxide.

In FIGS. 3 and 4 a preferred example of manufacturing a pre-compressed body 2 for use in a dressing 1, 1′ according to the present invention is schematically illustrated. A body 2 of resilient absorbent material, such as polyurethane foam, is in fully expanded state placed between two sheets 3A and 3B. Sheet 3B is placed on a stationary plate 9 having a similar shape as the cross-section of body 2 so that the contours of body 2 and plate 9 coincidence. On the upper side of sheet 3A, a plate 10 having a similar shape is arranged. Plate 10 is movable towards plate 9. Thereafter, plate 10 is moved towards plate 9 with a predetermined force F and a predetermined distance corresponding to the degree of compression desired for body 2. During the compression of body 2 air from the body escapes in sideways directions. When body 2 has been compressed to the desired degree, a tool for fastening sheets 3A and 3B together in the region outside the contours of plates 9,10 and thereby outside the contour of the compressed body presses the sheets 3A,B against each other in said region. In FIG. 3 only parts 11 of said tool is schematically illustrated. The fastening of the sheets to each other can be made in any suitable way such as by heat welding or gluing. If gluing is used, glue is applied to one or both of said sheets in said region before the sheets are pressed against each other. In FIG. 4, the configuration of the pre-compressed body 2 in its casing 3 is shown. Since the casing 3 is air-tight, no air can leak into the space inside the casing and the pre-compressed body 2 is thus prevented from expanding.

In the illustrated example sheets 3A and 3B are separate from each other but it is possible to instead use one sheet which is folded over the body 2 before compression thereof. In such a case, the tool for fastening the edges of the folded sheet together in the region outside the pre-compressed body 2 need not extend over the fold of the sheet.

In the example illustrated in FIGS. 3 and 4, the entire sheet 3B is preferably of soluble material, such as a film of polyvinyl alcohol whereas the sheet 3A can be of any air-tight material that can be affixed to the material of sheet 3B. It is possible to have both sheet 3A,B made of soluble material. If the casing 3 is constituted by a folded sheet, this is preferably of a soluble material even if it is possible to make an opening corresponding to the cross-section or at least to a major portion of the cross-section of body 2 in such a sheet and thereafter attach a piece of soluble material covering such an opening to the sheet. This should be done before the compression procedure described above takes place. It is possible, but not preferred, to provide an opening in sheet 3B in the same way and provide this opening with a cover of soluble material.

In the illustrated example of a manufacturing method for a pre-compressed body, the plates 9,10 and the fastening tool are dimensioned to coincide with the cross-section of the body 2. However, in order to ensure that the plates cover the whole area of the body 2 and allow a small misalignment of the body 2 relative to the plates 9,10 the plates might be slightly larger than the cross-section of body 2.

By providing a pre-compressed body in a separate casing as a unit in a wound dressing according to the present invention, a production line for the manufacture of dressings having wound pads of conventional design can be used for the manufacturing of dressings according to the present invention without change of equipment used or addition of new equipment. Neither will the production rate be slowed down by a compression step in the production line, which is especially important in continuous production lines. Moreover, the manufacturing of such a pre-compressed body will only need equipment of simple construction and can thus be produced in a cost-effective manner.

A wound dressing according to the present invention has many advantages compared with conventional island dressings. Firstly, the pressure difference between atmospheric pressure and the sub-pressure created presses the wound pad tightly against the wound bed so that a good contact between wound pad and the wound bed is ensured. As a matter of fact, said pressure difference will assure a better fixation of the wound dressing as a whole. The sub-pressure created above the wound bed will also ensure a better removal of exudate from the wound bed and also improve the healing of the wound.

In comparison with existing NPT-dressings, a wound dressing according to the present invention is less spacious, needs no electric or manual pump, makes no sound and is easier to use. A wound dressing according to the present invention is also of the disposable type and needs no service or cleaning. Moreover, it is a closed system with no external air-flow and consequently no external tubing which makes it very easy to use and to apply and also reduces the risk for cross-infection in comparison with reusables. A wound dressing according to the present invention has fewer parts than existing NPT-dressings and is thus easier to store and handle and can also be manufactured at a lower cost.

The described embodiments can of course be modified without leaving the scope of invention. The shapes of the wound dressings can for example be different than shown and described with reference to the figures. Other adhesives and materials used for known film dressings than stated above can be used. So called superabsorbent particles can be mixed within the pre-compressed bodies or be present in additional layers on top of the pre-compressed compressed bodies in order to further enlarge the liquid storing capacity of the wound dressings. The wound pad can consist of several layers. The scope of invention shall therefore only be restricted by the content of the enclosed patent claims.

1. A wound dressing including an outer layer of air-impermeable material and means for attaching the dressing to the skin surrounding a wound, a pre-compressed body of resilient material, said body creating a sub-pressure after attaching the dressing to a wound, wherein the pre-compressed body is enclosed in a casing of air-tight material which is separate from the outer layer, whereby at least a portion of said casing is made of a soluble material, which dissolves after contact with liquid exudates.

2. The dressing according to claim 1, wherein the pre-compressed body has the ability to directly or indirectly suck liquid from a wound bed.

3. The dressing according to claim 2, wherein the pre-compressed body constitutes the wound pad of the dressing.

4. The dressing according to claim 2, wherein the wound pad is constituted of several layers of which the pre-compressed body constitutes one of these layers.

5. The dressing according to claim 2, wherein the pre-compressed body is made of resilient foam.

6. The dressing according to claim 5, wherein the pre-compressed body is made of viscoelastic foam.

7. The dressing according to claim 1, wherein the dressing is stored in a vacuum package.

8. The dressing according to claim 1, wherein the casing at least on the side thereof, which is turned against a wound bed when the dressing is used, is made of soluble material.

9. The dressing according to claim 8, wherein the soluble material in the casing consists of polyvinyl alcohol.

10. The dressing according to claim 1, wherein the pre-compressed body enclosed in its casing is disposed outside the outer layer of the dressing and the unit of pre-compressed body and casing is enclosed in a second layer of air-tight material, which is releasably attached to the outer layer in a region around an opening in the outer layer and the second layer.

11. A combination of a first wound dressing including a wound pad and a cover layer of air-tight material and a second wound dressing according to claim 1, which is attached on top of the first wound dressing in a region thereof surrounding a hole in the cover layer of the first wound dressing.
